(19) United States
(12) Patent Application Publication
(10) Pub. No.: US 2025/0282696 A1
(43) Pub. Date: Sep. 11, 2025

(54) PROCESS FOR CONVERTING NAPHTHA WITH CRACKED RECYCLE

(71) Applicant: UOP LLC, Des Plaines, IL (US)

(72) Inventors: Robert Szczesniak, Chicago, IL (US); Gregory A. Funk, Carol Stream, IL (US)

(21) Appl. No.: 19/064,357

(22) Filed: Feb. 26, 2025

Related U.S. Application Data
(60) Provisional application No. 63/561,696, filed on Mar. 5, 2024.

(51) Int. Cl. C07C 4/04; C07C 7/00; C10G 9/36

(52) U.S. Cl. CPC C07C 4/04; C07C 7/005; C10G 9/36; C10G 2300/1037; C10G 2400/20

(57) ABSTRACT

A process for converting naphtha is disclosed. The process comprises contacting a naphtha stream and a hydrogen stream with a catalyst in a reactor to produce a paraffinic stream. The ethane present in the paraffinic stream is converted into ethylene in a steam cracking unit. An ethylene stream is separated from a C3+ hydrocarbon stream and the C3+ hydrocarbon stream is recycled to the reactor.

### FIELD

The field is the conversion of naphtha to paraffins. The field may particularly relate to converting naphtha to paraffins for producing light olefins.

### BACKGROUND

Light olefin production is vital to the production of sufficient plastics to meet worldwide demand. Paraffin dehydrogenation (PDH) is a process in which light paraffins such as propane and butane can be dehydrogenated to make propylene and butylene, respectively. Dehydrogenation is an endothermic reaction which requires external heat to drive the reaction to completion.

Fluid catalytic cracking (FCC) is another endothermic process that can be tuned to produce substantial propylene. However, not every FCC unit is tuned to make substantial propylene. Also, high propylene FCC units do not make much ethylene; less than 1% of global ethylene supply comes from FCC.

The great bulk of the ethylene consumed in the production of plastics and petrochemicals such as polyethylene is produced by the thermal cracking of hydrocarbons. Steam is usually mixed with the feed stream to the cracking furnace to reduce the hydrocarbon partial pressure and enhance olefin yield and to reduce the formation and deposition of carbonaceous material in the cracking reactors. The process is therefore often referred to as steam cracking or pyrolysis. Ethane oxidative dehydrogenation is a newer catalytic process for converting ethane to ethylene which can be conducted at lower temperatures with lower carbon oxide emissions than steam cracking.

Two types of feeds are typically used for steam cracking. Ethane feed is used in regions where light hydrocarbon gases are prevalent. In regions where gas is not abundant, naphtha feed is employed for steam cracking. Pyrolytic naphtha cracking has long set the price in the ethylene industry due to higher production cost versus pyrolytic ethane cracking. The world does not currently produce enough ethane to supply the growing demand for ethylene. Therefore, regions lacking ethane supply such as Asia and Europe rely mainly on naphtha cracking to supply ethylene. Naphtha cracking yields only about 30%-35% ethylene with the balance including both relatively high-value by-products comprising propylene, butadiene, and butene-1 and relatively low value by-products comprising pyoil, pygas, and fuel gas. Additional pressures on naphtha cracking including minimum production requirements and environmental concerns have led to the withholding of government approvals in certain regions such as China.

The ethane charged crackers produce some by-products. However, the amount of the by-products from ethane fed crackers is relatively small and, in many cases, may not justify the investment to recover and purify some of the by-products.

The ethylene industry needs a more efficient, economical and environmentally friendly route to light olefins from naphtha feeds.

### BRIEF SUMMARY

A process for converting naphtha is disclosed. The process comprises contacting a naphtha stream and a hydrogen stream with a catalyst in a reactor to produce a paraffinic stream. The ethane present in the paraffinic stream is converted into ethylene in a steam cracking unit. An ethylene stream in an embodiment from the steam cracking unit is separated from a C3+ hydrocarbon stream and the C3+ hydrocarbon stream is recycled to the reactor. The process with C3+ hydrocarbon recycle provides a higher yield of ethylene than a traditional steam cracker.

### BRIEF DESCRIPTION OF THE DRAWINGS

FIG. 1 is a schematic drawing of a process for converting naphtha in accordance with an exemplary embodiment of the present disclosure.

FIG. 2 is a schematic drawing of a process for converting naphtha in accordance with another exemplary embodiment of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

As used herein, the term “predominant” or “predominate” means greater than 50%, suitably greater than 75% and preferably greater than 90%.

The term “Cx” is to be understood to refer to molecules having the number of carbon atoms represented by the subscript “x”. Similarly, the term “Cx-” refers to molecules that contain less than or equal to x and preferably x and less carbon atoms. The term “Cx+” refers to molecules with more than or equal to x and preferably x and more carbon atoms.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take a main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

### DETAILED DESCRIPTION

In the proposed process, a naphtha feed stock is primarily charged to a “Naphtha to Ethane and Propane” (NEP) unit to convert naphtha into desirable ethane and propane along with less desirable methane. The produced ethane stream is fed to a steam cracking unit to convert ethane into ethylene. The steam cracking unit also produces by-products which are separated in a recycle stream from the steam cracking unit. The byproduct components are recovered in a single collective recycle stream which is recycled back to the NEP reactor. The recycling byproduct components to the NEP reactor produces additional ethane for the steam cracking unit, resulting in increased ethylene production from the same amount of fresh feed.

Turning to FIGURE, a process for converting naphtha 101 is shown. A naphtha stream in line 102 may be combined with a hydrogen stream in line 103 to provide a charge stream in line 104. The charge stream in line 104 may be heated and charged to a naphtha to ethane and propane (NEP) reactor 120 to be contacted with an NEP catalyst. As described later in detail, a recycle stream in line 144 may also be combined with the naphtha stream in line 102 and passed to the reactor 120 in the charge line 104. The naphtha stream may comprise C4 to C12 hydrocarbons preferably having a T10 between about −10° C. and about 60° C. and a T90 between about 70 and about 180° C. The naphtha feed stream may comprise normal paraffins, iso-paraffins, naphthenes, and aromatics. The naphtha feed stream may comprise hydrocarbons having any of 3 to 12 carbon atoms per molecule. Suitably, the naphtha feed stream may comprise hydrocarbons having any of 3 to 8 carbon atoms per molecule or any of 3 to 6 carbons per molecule. The naphtha stream may be heated to a reaction temperature of about 300° C. to about 600° C., suitably between about 325° C. and about 550° C., and preferably between about 350° C. and about 525° C. Weight space velocity should be about 0.3 to about 20 hr⁻¹, suitably between about 0.5 and about 10 hr⁻¹ and preferably between about 1 to about 4 hr⁻¹. A total pressure should be about 0.1 to about 3 MPa (abs), preferably no more than 2 MPa (abs). At these conditions, C2-C4 yield is consistently in an excess of 80 wt %, while methane yield is less than about 10 wt %, suitably below about 8 wt % and typically below about 6 wt % and preferably no more than 5 wt %. Under these conditions, ethane may comprise more than about 60 wt % of the total C2 to C3 in the NEP reactor 120. In some embodiment, ethane can comprise about 20% to about 60% of the total C2 to C3 and/or C2 to C4 produced in the NEP reactor 120.

The hydrogen-to-hydrocarbon molar ratio is important to producing ethane and propane. The hydrogen-to-hydrocarbon ratio should be about 0.3 to about 15 and preferably about 0.5 to about 5. In a further embodiment, the hydrogen-to-hydrocarbon molar ratio may typically be no more than 5, suitably be no more than 3 and preferably be no more than 2. Low hydrogen-to-hydrocarbon ratio promotes desired reaction kinetics which are initiated with dehydrogenation. Hydrogen-to-hydrocarbon ratio may range from about 50% to about 500%, suitably no more than 300% and preferably no more than 200% of stoichiometric requirements to convert naphtha molecules to ethane and/or propane. The molar ratio of hydrogen to hydrocarbon depends on the feed type including paraffin, olefin, naphthene or aromatics, the feed molecular carbon number, and the desired product between predominantly ethane, predominantly propane or ethane and propane of comparable abundance.

The NEP catalyst for converting naphtha to ethane and propane may contain a molecular sieve comprising large or medium pore mouths, that is, comprising 10 or 12 member rings, respectively. Examples of suitable molecular sieves include MFI, MEL, MFI/MEL intergrowth, MTW, TUN, UZM-39, IMF, UZM-44, UZM-54, MWW, UZM-37, UZM-8, UZM-8HS. Examples of suitable molecular sieves further include FER, AHT, AEL (SAPO-11), AFO (SAPO-41), MRE, MFS, EUO-1, TON (ZSM-22), MTT (ZSM-23) and UZM-53. Additional molecular sieves with larger pores include FAU, EMT, FAU/EMT intergrowth, UZM-14, MOR, BEA, UZM-50, MTW, ZSM-12. Additional examples include MSE and UZM-35.

MFI is a suitable NEP catalyst. It will be appreciated that ZSM-5 is an MFI-type aluminosilicate zeolite belonging to the pentasil family of zeolites and having a chemical formula of NaₙAlₙSi₉₆-nO₁₉₂·16H₂O (0<n<10). In various embodiments, the ZSM-5 zeolite may comprise a silica-to-alumina molar ratio of about 20 to about 1000, about 20 to about 800, about 20 to about 600, about 20 to about 400, about 20 to about 200 or about 20 to about 80. In various embodiments, the ZSM-5 zeolite may comprise a crystal size in the range of about 10 to about 600 nm, about 20 to about 500 nm, about 30 to about 450, about 40 about to 400 nm, or about 50 to about 300 nm.

The NEP catalyst may comprise a bound zeolite. The binder may comprise an oxide of aluminum, silicon, zinc, titanium, zirconium and mixtures thereof. The binder may comprise a phosphate in the binder or a phosphate of the forenamed oxide binder materials. Preferably, the binder is a silicon oxide. The MFI zeolite may be supported in a silicon oxide containing binder or an alumina containing binder such as aluminum phosphate.

MFI zeolite slurry may be first mixed with a binder in the form of colloidal suspension (sol) and gelling reagent and then dropped into hot oil to make spheres controlled to produce about 1/32-inch to about ⅛-inch diameter calcined supports. Alternatively, the zeolite may be mixed with a silicon oxide containing binder and extruded to 1/32 to ¼-inch diameter extrudates. Extrudates may be washed with ammonia to remove sodium ions from the zeolite, dried and calcined to remove the organic structural directing agent (OSDA) from the synthesized zeolite. Optionally, the calcined support may be ammonium-ion exchanged using an ammonium nitrate solution to remove residual sodium ions and dried at about 110° C.

The NEP catalyst comprises a metal on the catalyst. The metal may comprise a transition metal. In a further example, the metal may comprise platinum, palladium, iridium, rhenium, ruthenium and mixtures thereof. The metal may be a noble metal. A modifier metal may also be included on the catalyst. The modifier metal may include tin, germanium, gallium, indium, thallium, zinc, silver and mixtures thereof. The modifier metal should be more concentrated on the binder than on the zeolite. About 0.01 to about 5 wt % of each transition metal and the modifier metal may be on the catalyst.

Metal may be incorporated into the binder by evaporative impregnation. A solution of platinum such as tetraamine platinate nitrate or chloroplatinic acid may be contacted with the bound spherical or extrudate supports which have been calcined and ion-exchanged in a rotary evaporator, followed by drying and oxidation.

The NEP catalyst comprises a metal on the bound spherical or extrudate supports of the catalyst. Preferably, more of the metal is on the binder than on the zeolite. At least about 60 wt %, suitably at least about 70 wt %, preferably at least about 80 wt % and most preferably at least about 90 wt % of the metal is on the binder. The zeolite and/or the entire NEP catalyst is steam oxidized to drive the metal off the zeolite. Steaming is preferably effected after the metal is added to the catalyst. The dried, impregnated spherical or extrudate supports may be steam oxidized in air for sufficient time to provide NEP catalysts. Steam oxidation in air at a temperature of about 500° C. to about 650° C. and about 5 mol % to about 30 mol % steam for about 1 to about 3 hours may be suitable.

The NEP catalysts must be reduced to activate them for catalyzing the NEP reaction. For example, the catalyst may be reduced in flowing hydrogen at about 500 to about 550° C. for about 3 hours before contacting feed.

After paraffin conversion, a reactor effluent stream comprising paraffins is discharged from the NEP reactor 120 in an effluent line 122. The reactor effluent stream in line 122 may be a light paraffinic stream. The reactor effluent stream in line 122 may comprise at least about 40 wt % ethane or at least about 40 wt % propane or at least about 70 wt % ethane and propane and preferably at least about 80 wt % ethane and propane. The ethane to propane ratio can range from about 0.1 to about 5. In an aspect, the ethane and propane may comprise at least about 70% of the non-aromatics present in the reactor effluent stream in line 122.

The reactor effluent stream in line 122 may be passed to a steam cracking unit 130 to convert the ethane present in the reactor effluent stream to ethylene. Aromatics may be separated from the reactor effluent stream in line 122 before passing the effluent stream to the steam cracking unit 130. In an aspect, the reactor effluent stream in line 122 may be passed to a separation unit 123 to separate aromatics from the reactor effluent stream before passing the reactor effluent stream to the steam cracking unit 130. The reactor effluent stream in line 122 may be cooled and fed to a separation unit 123. The separation unit 123 may be a fractionation column or a series of fractionation columns and other separation units that may separate the reactor effluent stream in line 122 into the hydrogen stream in line 125, an ethane containing stream in line 124, a propane containing stream in line 126 and the heavy stream comprising aromatics in line 127.

The separation unit 123 may comprise a demethanizer column that separates the light paraffins into a gas stream in an overhead line and a C2+ paraffin stream in a bottoms line. The gas stream may be sent to a hydrogen purification unit such as a PSA unit to recover hydrogen in line 125 for recycle to the NEP reactor 120. Remaining methane in line 121 from the hydrogen purification unit may be used for fuel gas. The C2+ paraffin stream may then be fed to a deethanizer column to produce the ethane containing stream in line 124 and a C3+ paraffin stream. The C3+ paraffin stream may then be fed to a depropanizer column to produce the propane containing stream in line 126 and a heavy stream comprising aromatics in the recycle line 127 which may comprise C4+ hydrocarbons. The heavy stream in line 127 may be recycled to the NEP reactor 120. A net aromatics stream may be taken in line 129. The separation unit 123 may take other forms.

For example, the separation unit 123 may omit the demethanizer column and the paraffin stream may feed a deethanizer column which produces a C2− stream in a deethanizer overhead line. The C2− stream can be separated in the hydrogen purification unit to recover a hydrogen stream in line 125 while residual ethane and methane from the hydrogen purification unit can comprise or supplement the ethane stream in line 124. The hydrogen purification unit may comprise a membrane unit and the hydrogen recovered from the membrane unit may be further purified in an absorption column before it is recycled to the NEP reactor 120 in line 125. In an additional alternative, the C2− stream from the deethanizer column may be charged to an ethylene producing unit such as the steam cracking unit 130 in which ethane is converted to ethylene but methane and hydrogen rides through inertly to be recovered in a downstream ethylene recovery unit.

The ethane containing stream in line 124 is passed to the steam cracking unit 130. In an embodiment, the propane containing stream in line 126 may be passed to the steam cracking unit 130 so no propane product stream is taken from the separation unit 123. In another aspect, the ethane containing stream in line 124 and the propane containing stream in line 126 may be combined and a combined stream may be passed to the steam cracking unit 130.

In a preferred embodiment, the propane stream in line 126 may be taken from the separation unit 123 and charged to a propylene producing unit 150 in which propane in the propane stream is converted into propylene. The propylene producing unit 150 may be a paraffin dehydrogenation (PDH) unit. In another embodiment, the propane stream in line 126 may be recycled to the NEP reactor 120 via line 144.

In the steam cracking unit 130, ethane from the reactor effluent stream in line 124 is cracked under steam in a furnace to produce a steam cracked effluent stream. Ethylene is separated from the steam cracked effluent stream and taken in line 132. The ethane may be charged to the steam cracking unit 130 in the gas phase. The steam cracking unit 130 may preferably be operated at a temperature of about 750° C. (1382° F.) to about 950° C. (1742° F.). The cracked stream exiting the furnace of the steam cracking unit 130 may be in a superheated state. One or more quench columns, or other devices known in the art, but preferably an oil quench column and/or a water quench column, may be used for quenching or separating the cracked stream into a plurality of cracked streams. The steam cracking unit 130 may further comprise additional distillation columns, amine wash columns, compressors, expanders, etc. to separate the steam cracked effluent stream into cracked streams rich in individual light olefins the most predominant of which is the ethylene stream in line 132. The ethylene stream may comprise a yield of at least about 75 wt %, preferably at least about 80 wt %, ethylene based on the ethane stream in line 122. Other components in the steam cracked effluent stream exiting the steam cracking unit 130, may include hydrogen, methane, propylene, butene, and pyrolysis gas.

The ethylene stream in line 132 from the steam cracking unit 130 may be recovered or transported to polymerization plants, chemical plants or exported. Product recovery of at least 50 wt %, typically at least 60 wt % and suitably at least 70 wt % of valuable ethylene is achievable from the steam cracking unit 130 based on the ethane stream in line 122.

Byproducts from the steam cracking unit comprise C3+ hydrocarbons. Usually, for a steam cracking unit the C3-C6 byproducts are recycled back to the steam cracking unit 130 or further processed to obtain product streams for other uses. Applicants found that these C3-C6 hydrocarbons, particularly paraffins may not be good recycle feeds to the cracker for producing incremental ethane and increasing the overall ethylene yield. The low-quality heavy by products significantly reduce the yield efficiency of the steam cracking unit. Recycling the C3-C6 byproducts back to the steam cracking unit resulted in similar, low ethylene production as exhibited for fresh feed of C3-C6 hydrocarbons. However, this same fresh feed of C3-C6 hydrocarbons with recycle of steam cracked C3-C6 hydrocarbons to the steam cracking unit produces less hydrogen, more fuel gas, and more heavy (C6+) components. The coke rate in the coil is likely to be negatively impacted as well, adding operational negatives to the recycle case.

Applicants found that taking C3+ hydrocarbons from the steam cracking unit 130 in one stream and recycling them back to the NEP reactor 120 produces better incremental ethane and increasing the overall ethylene yield of the steam cracking unit 130. The recycling of C3-C6 hydrocarbons to the NEP reactor 120 provides a better steam cracker feed which increases high value product ethylene yield from the steam cracking unit 130 and removes additional processing requirements. This reduces the ratio of feedstock purchase per mass of ethylene product and reduces the capital investment required and carbon footprint per mass of ethylene product while increasing the economic return. Beyond simplifying the separation steps of the steam cracking unit 130, applicants found that the recycling of C3-C6 hydrocarbons to the NEP reactor 120 turned much of the byproducts stream into a high-quality ethane feed to the steam cracking unit 130 and boosts ethylene production of the steam cracking unit 130 by about 5% from the same fresh feed with C3-C6 hydrocarbon recycle.

In accordance with the present disclosure, the C3+ hydrocarbons comprising propane, propylene, butane, butadiene, butene, mixed C5, and C6 non-aromatics components are taken in a collective C3+ hydrocarbon stream in line 133 and recycled to the NEP reactor 120. In an aspect the C3+ hydrocarbon stream in line 133 may be combined with the naphtha stream in line 102 and the hydrogen stream in line 103 and charged to the reactor 120 in the charge line 104.

In an aspect, the additional treatment such as a complete or selective hydrogenation may be provided to the C3+ hydrocarbon stream in line 133 to additionally improve performance. In an embodiment, the C3+ hydrocarbon stream in line 133 may be passed to a hydrogenation unit 140. In an exemplary embodiment, the hydrogenation unit 140 is a selective hydrogenation unit. In the selective hydrogenation unit 140, the C3+ hydrocarbon stream in line 133 is contacted with a selective hydrogenation catalyst.

Selective hydrogenation catalyst may comprise an alumina support material preferably having a total surface area greater than 150 m²/g, with most of the total pore volume of the catalyst provided by pores with average diameters of greater than 600 angstroms and containing surface deposits of about 1.0 to 25.0 wt % nickel and about 0.1 to 1.0 wt % sulfur such as disclosed in U.S. Pat. No. 4,695,560. Spheres having a diameter between about 0.4 and 6.4 mm ( 1/64 and ¼ inch) can be made by oil dropping a gelled alumina sol. The alumina sol may be formed by digesting aluminum metal with an aqueous solution of approximately 12 wt % hydrogen chloride to produce an aluminum chloride sol. The nickel component may be added to the catalyst during the sphere formation or by immersing calcined alumina spheres in an aqueous solution of a nickel compound followed by drying, calcining, purging and reducing. The nickel containing alumina spheres may then be sulfided.

The selective hydrogenation is normally performed at relatively mild hydrogenation conditions. These conditions will normally result in the hydrocarbons being present as liquid phase materials. The reactants will normally be maintained under the minimum pressure sufficient to maintain the reactants as liquid phase hydrocarbons which allows the hydrogen to dissolve into the hydrocarbonaceous hydrogenation feed. A broad range of suitable operating pressures therefore extends from about 376 kPa (40 psig) to about 5617 kPa (800 psig), with a pressure between about 445 kPa (50 psig) and about 2169 kPa (300 psig) being preferred. A relatively moderate temperature between about 25° C. (77° F.) and about 350° C. (662° F.) should be employed. Preferably, the temperature of the hydrogenation zone is maintained between about 30° C. (122° F.) and about 200° C. (392° F.). The liquid hourly space velocity of the reactants through the selective hydrogenation catalyst should be at least about 1.0 hr⁻¹. Preferably, it is above about 5.0 and more preferably it is between about 5.0 and about 35.0 hr⁻¹. The ratio of hydrogen to diolefinic hydrocarbons maintained within the selective hydrogenation zone is an important variable. The amount of hydrogen required to achieve a desired conversion is believed to be dependent upon both reactor temperature and the molecular weight of the feed hydrocarbons. To avoid the undesired saturation of a significant amount of monoolefinic hydrocarbons, there should be less than 2 times the stoichiometric amount of hydrogen required for the selective hydrogenation of the diolefinic hydrocarbons which are present in the liquid phase process stream to monoolefinic hydrocarbons. Preferably, the mole ratio of hydrogen to diolefinic hydrocarbons in the material entering the bed of selective hydrogenation catalyst is maintained between 1:1 and 1.8:1. In some instances, it may be desirable to operate with a less than stoichiometrically required amount of hydrogen, with mole ratios down to 0.75:1 being acceptable. The optimum set of conditions will of course vary depending on such factors as the composition of the feed stream and the degree of saturation of diolefinic hydrocarbons which is desired.

The selective hydrogenation reactor is preferably a cylindrical fixed bed of catalyst through which the reactants move in a vertical direction. It is preferred that the reactants flow upward through the reactor as this provides good mixing. The hydrogenation catalyst may be present within the reactor as pellets, spheres, extrudates, irregular shaped granules, etc. To employ the hydrogenation catalyst, the reactants would be preferably brought to the desired inlet temperature of the reaction zone, admixed with hydrogen and then passed into and through the reactor. Alternatively, the reactants may be admixed with the desired amount of hydrogen in line 142 and then heated to the desired inlet temperature. In either case, the effluent of the hydrogenation reactor may be passed into a hydrogen recovery facility for the removal of residual hydrogen before proceeding further in the process. Hydrogen may be removed by flashing the hydrogenation effluent stream to a lower pressure or by passing the effluent stream into a stripping column. Otherwise, no residual hydrogen recovery may be necessary if the residual hydrogen concentration in the hydrogenation effluent is acceptable. The effluent from the selective hydrogenation reactor will preferably have less than 100 ppm of diolefins.

In an aspect, the selective hydrogenation step may be optionally used. In an exemplary embodiment, the selective hydrogenation reactor may be omitted if the concentration of diolefins in the C3+ hydrocarbon stream in line 133 is below 100 ppm.

A hydrogenated effluent stream may be taken in line 144 from the selective hydrogenation unit 140. The hydrogenated effluent stream in line 144 is recycled to the NEP reactor 120. The hydrogenated effluent stream in line 144 may be combined with the naphtha stream in line 102 and the hydrogen stream in line 103 and passed to the NEP reactor 120 in the charge line 104. In an embodiment, the C3+ hydrocarbon stream in line 133 forms about 0.5 wt % to about 10 wt % of the charge stream in the charge line 104 to the reactor 120.

In an embodiment, C6+ hydrocarbons including aromatics may be separated from the steam cracked effluent in the steam cracking unit 130. The C6+ hydrocarbons may include benzene, toluene, xylenes, ethyl benzene, and C9+ hydrocarbons. The C6+ hydrocarbons are separated from the steam cracking unit 130 in line 138 to provide an aromatic free C3+ hydrocarbon stream in line 133.

A recycle stream comprising hydrogen is separated from the steam cracking unit 130 in line 131 and recycled to the NEP reactor 120. In an embodiment, the recycle stream in line 131 is combined with the hydrogen stream in line 125 to provide a combined recycle hydrogen stream in line 128 which is passed to the NEP reactor 120. Methane can be removed from the steam cracking unit 130 in line 135 and used as fuel gas.

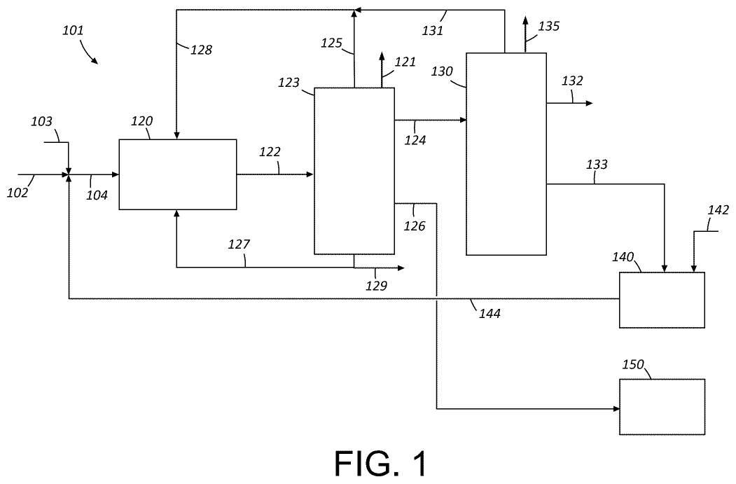

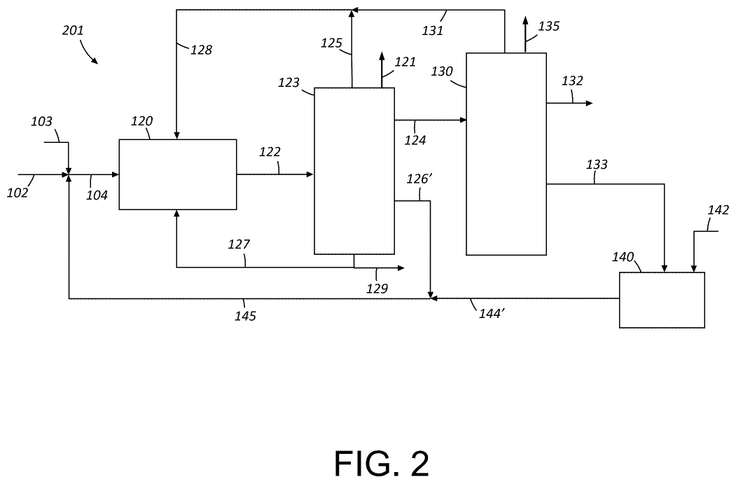

FIG. 2 shows an alternative embodiment to the embodiment of FIG. 1 which employs a dedicated propane to ethane reactor 120. Elements in FIG. 2 with the same configuration as in FIG. 1 will have the same reference numeral as in FIG. 1. Elements in FIG. 2 which have a different configuration as the corresponding element in FIG. 1 will have the same reference numeral but designated with a prime symbol (′). The configuration and operation of the embodiment of FIG. 2 is essentially the same as in FIG. 1 with the following exceptions.

As shown in FIG. 2, the propane containing stream is withdrawn from the separation unit 123 in line 126′ and recycled back to the reactor 120. In this embodiment, the propane containing stream is not separately processed or taken as a product stream. The propane containing stream in line 126′ may be passed to the reactor 120 in the charge line 104. In an aspect, the propane containing stream in line 126′ may be combined with the hydrogenated effluent stream in line 144′ to provide a mixed stream in line 145. The mixed stream in line 145 may be combined with the naphtha stream in line 102 and the hydrogen stream in line 103 and charged to the reactor 120 in the charge line 104. In an embodiment without the hydrogenation reactor 140, the propane containing stream in line 126′ is combined with the C3+ hydrocarbon stream in line 133 to provide mixed stream in line 145 that is recycled to the NEP reactor 120. With these exceptions, the rest of the process is the same as described for FIG. 1.

The foregoing disclosure provides a process for converting naphtha to ethane and propane feed with maximized conversion to ethylene and propylene in ethylene and propylene producing units, respectively.

### Example

A comparative study was performed to compare the present process with the typical steam cracker operations for ethylene yield. The process with C3+ hydrocarbon recycle to the NEP reactor was compared with a typical steam cracker operation with no recycle stream and another typical steam cracker with recycle back to the steam cracker. The parameters and the results of the study are shown in the Table. Coil outlet refers to the outlet of the steam cracking reactor. Net product weight fraction is different from the coil outlet weight fraction due to internal recycle of the unconverted material in the steam cracking unit.

TABLE

| | Base Case 1 | | | | | |
|---|---|---|---|---|---|---|
| | No Recycle Stream | | Base Case 2 | | Present process | |
| | from Steam | | Recycle to Steam | | Recycle to NEP | |
| | Cracking Unit | | Cracking Unit | | Reactor | |
| | Coil | Net | Coil | Net | Coil | Net |
| | outlet, | product, | outlet, | product, | outlet, | product, |
| Component | wt % | wt % | wt % | wt % | wt % | wt % |
| Hydrogen | 3.8 | 5.5 | 2.8 | 4.5 | 3.8 | 6.3 |
| Methane | 6.2 | 9.7 | 7.2 | 11.7 | 6.2 | 10.2 |
| Acetylene | 0.4 | 0.7 | 0.4 | 0 | 0.4 | 0 |
| Ethylene | 51.5 | 77.4 | 48.2 | 77.5 | 51.5 | 82.6 |
| Ethane | 33.6 | 0 | 31.4 | 0 | 33.6 | 0 |
| Propylene | 1.1 | 1.6 | 1.0 | 0 | 1.1 | 0 |
| Propane | 0.3 | 0.5 | 0.3 | 0 | 0.3 | 0 |
| Other C3 | 0 | 0.1 | 0 | 0 | 0 | 0 |
| Butadiene | 1.4 | 2.1 | 1.3 | 0 | 1.4 | 0 |
| Butane | 0.4 | 0.6 | 0.4 | 0 | 0.4 | 0 |
| Butene | 0.3 | 0.4 | 0.3 | 0 | 0.3 | 0 |
| Mixed C5 | 0.4 | 0.5 | 2.6 | 0 | 0.4 | 0 |
| C6 non- | 0.1 | 0.1 | 0.3 | 0 | 0.1 | 0 |
| aromatics | | | | | | |
| Benzene | 0.4 | 0.7 | 3.3 | 5.3 | 0.4 | 0.8 |
| Toluene | 0.1 | 0.1 | 0.3 | 0.5 | 0.1 | 0.1 |
| Xylenes | 0 | 0 | 0 | — | 0 | 0 |
| Ethyl | 0 | 0 | 0 | — | 0 | 0 |
| benzene | | | | | | |
| C9+ | 0 | 0 | 0.2 | 0.5 | 0 | 0 |

The results in the Table demonstrate that the process with C3+ hydrocarbon recycle to the NEP reactor produces a higher ethylene yield in terms of net product wt %. The ethylene yield from the C3+ hydrocarbon recycle to NEP reactor was over 5 wt % greater than the typical steam cracking operation.

### Specific Embodiments

While the following is described in conjunction with specific embodiments, it will be understood that this description is intended to illustrate and not limit the scope of the preceding description and the appended claims.

A first embodiment of the present disclosure is a process for converting naphtha, comprising contacting a naphtha stream and a hydrogen stream with a catalyst in a reactor to produce a paraffinic stream; converting ethane in the paraffinic stream into ethylene in a steam cracking unit; separating an ethylene stream from a C3+ hydrocarbon stream; and recycling the C3+ hydrocarbon stream to the reactor. An embodiment of the present disclosure is one, any or all of prior embodiments in this paragraph up through the first embodiment in this paragraph, wherein the C3+ hydrocarbon stream comprises C3 to C6 hydrocarbons. An embodiment of the present disclosure is one, any or all of prior embodiments in this paragraph up through the first embodiment in this paragraph, wherein the C3+ hydrocarbon stream comprises non-aromatic hydrocarbons. An embodiment of the present disclosure is one, any or all of prior embodiments in this paragraph up through the first embodiment in this paragraph, further comprising separating a steam cracked effluent stream into the ethylene stream and the C3+ hydrocarbon stream. An embodiment of the present disclosure is one, any or all of prior embodiments in this paragraph up through the first embodiment in this paragraph further comprising mixing the C3+ hydrocarbon stream with the naphtha stream; and contacting the C3+ hydrocarbon stream, the naphtha stream and the hydrogen stream with the catalyst in the reactor. An embodiment of the present disclosure is one, any or all of prior embodiments in this paragraph up through the first embodiment in this paragraph wherein the C3+ hydrocarbon stream forms 0.5 wt % to about 10 wt % of a charge stream to the reactor. An embodiment of the present disclosure is one, any or all of prior embodiments in this paragraph up through the first embodiment in this paragraph further comprising hydrogenating the C3+ hydrocarbon stream to produce a hydrogenated stream; and recycling the hydrogenated stream to the reactor. An embodiment of the present disclosure is one, any or all of prior embodiments in this paragraph up through the first embodiment in this paragraph further comprising separating an ethane rich stream from the paraffinic stream; and charging the ethane rich stream to the steam cracking unit. An embodiment of the present disclosure is one, any or all of prior embodiments in this paragraph up through the first embodiment in this paragraph further comprising separating aromatics from the paraffinic stream to produce a heavy stream comprising aromatics and a light paraffinic stream; and charging the light paraffinic stream to the steam cracking unit. An embodiment of the present disclosure is one, any or all of prior embodiments in this paragraph up through the first embodiment in this paragraph further comprising separating a hydrogen containing stream from the paraffinic stream in the steam cracking unit and recycling the hydrogen containing stream back to the contacting step. An embodiment of the present disclosure is one, any or all of prior embodiments in this paragraph up through the first embodiment in this paragraph wherein the naphtha stream and the hydrogen stream are contacted with a zeolitic catalyst in the reactor. An embodiment of the present disclosure is one, any or all of prior embodiments in this paragraph up through the first embodiment in this paragraph wherein all propane in the paraffinic stream is recycled to the reactor. An embodiment of the present disclosure is one, any or all of prior embodiments in this paragraph up through the first embodiment in this paragraph wherein propane is separated from the paraffinic stream in the steam cracking unit. An embodiment of the present disclosure is one, any or all of prior embodiments in this paragraph up through the first embodiment in this paragraph further comprising separating a propane stream from the paraffinic stream and recycling the propane stream to the to the reactor.

A second embodiment of the present disclosure is a process for converting naphtha comprising contacting a naphtha stream with a zeolitic catalyst in a reactor to produce a paraffinic stream; converting ethane in the paraffinic stream into ethylene in a steam cracking unit; separating a steam cracked effluent stream into a hydrogen stream, an ethylene stream, and a C3+ hydrocarbon stream; and recycling the C3+ hydrocarbon stream to the reactor. An embodiment of the present disclosure is one, any or all of prior embodiments in this paragraph up through the second embodiment in this paragraph, wherein the C3+ hydrocarbon stream comprises C3 to C6 non-aromatic hydrocarbons. An embodiment of the present disclosure is one, any or all of prior embodiments in this paragraph up through the second embodiment in this paragraph wherein the C3+ hydrocarbon stream forms 0.5 wt % to about 10 wt % of a charge stream to the reactor. An embodiment of the present disclosure is one, any or all of prior embodiments in this paragraph up through the second embodiment in this paragraph further comprising hydrogenating the C3+ hydrocarbon stream to produce a hydrogenated stream; and recycling the hydrogenated stream to the reactor.

A third embodiment of the present disclosure is a process for converting naphtha, comprising contacting a naphtha stream and a hydrogen stream with a catalyst in a reactor to produce a paraffinic stream; converting ethane in the paraffinic stream into ethylene in a steam cracking unit; separating a steam cracked effluent stream into an ethylene stream and a C3-C6 non-aromatic hydrocarbons stream; and recycling the C3-C6 non-aromatic hydrocarbons stream from the steam cracking unit to the reactor. An embodiment of the present disclosure is one, any or all of prior embodiments in this paragraph up through the second embodiment in this paragraph, wherein the C3-C6 non-aromatic hydrocarbons stream forms 0.5 wt % to about 10 wt % of a charge stream to the reactor. An embodiment of the present disclosure is one, any or all of prior embodiments in this paragraph up through the second embodiment in this paragraph, further comprising hydrogenating the C3-C6 non-aromatic hydrocarbons stream to produce a hydrogenated stream, and recycling the hydrogenated stream to the reactor.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for converting naphtha, comprising:
contacting a naphtha stream and a hydrogen stream with a catalyst in a reactor to produce a paraffinic stream;
converting ethane in said paraffinic stream into ethylene in a steam cracking unit;
separating an ethylene stream from a C3+ hydrocarbon stream; and
recycling said C3+ hydrocarbon stream to the reactor.

2. The process of claim 1, wherein said C3+ hydrocarbon stream comprises C3 to C6 hydrocarbons.

3. The process of claim 1, wherein said C3+ hydrocarbon stream comprises non-aromatic hydrocarbons.

4. The process of claim 1, further comprising separating a steam cracked effluent stream into said ethylene stream and said C3+ hydrocarbon stream.

5. The process of claim 1 further comprising:
mixing said C3+ hydrocarbon stream with said naphtha stream; and
contacting said C3+ hydrocarbon stream, said naphtha stream and said hydrogen stream with the catalyst in the reactor.

6. The process of claim 5 wherein said C3+ hydrocarbon stream forms 0.5 wt % to about 10 wt % of a charge stream to the reactor.

7. The process of claim 1 further comprising:
hydrogenating said C3+ hydrocarbon stream to produce a hydrogenated stream; and
recycling said hydrogenated stream to the reactor.

8. The process of claim 1 further comprising:
separating an ethane rich stream from said paraffinic stream; and
charging said ethane rich stream to the steam cracking unit.

9. The process of claim 1 further comprising:
separating aromatics from said paraffinic stream to produce a heavy stream comprising aromatics and a light paraffinic stream; and
charging said light paraffinic stream to the steam cracking unit.

10. The process of claim 1 further comprising separating a hydrogen containing stream from said paraffinic stream in the steam cracking unit and recycling said hydrogen containing stream back to the contacting step.

11. The process of claim 1 wherein all propane in said paraffinic stream is recycled to the reactor.

12. The process of claim 11 wherein propane is separated from said paraffinic stream in the steam cracking unit.

13. The process of claim 11 further comprising:
separating a propane stream from said paraffinic stream; and
recycling said propane stream to the to the reactor.

14. A process for converting naphtha comprising:
contacting a naphtha stream with a zeolitic catalyst in a reactor to produce a paraffinic stream;
converting ethane in said paraffinic stream into ethylene in a steam cracking unit;
separating a steam cracked effluent stream into a hydrogen stream, an ethylene stream, and a C3+ hydrocarbon stream; and
recycling said C3+ hydrocarbon stream to the reactor.

15. The process of claim 14, wherein said C3+ hydrocarbon stream comprises C3 to C6 non-aromatic hydrocarbons.

16. The process of claim 14 wherein said C3+ hydrocarbon stream forms 0.5 wt % to about 10 wt % of a charge stream to the reactor.

17. The process of claim 14 further comprising:
hydrogenating said C3+ hydrocarbon stream to produce a hydrogenated stream; and
recycling said hydrogenated stream to the reactor.

18. A process for converting naphtha, comprising:
contacting a naphtha stream and a hydrogen stream with a catalyst in a reactor to produce a paraffinic stream;
converting ethane in said paraffinic stream into ethylene in a steam cracking unit;
separating a steam cracked effluent stream into an ethylene stream and a C3-C6 non-aromatic hydrocarbons stream; and
recycling said C3-C6 non-aromatic hydrocarbons stream from the steam cracking unit to the reactor.

19. The process of claim 18 wherein said C3-C6 non-aromatic hydrocarbons stream forms 0.5 wt % to about 10 wt % of a charge stream to the reactor.

20. The process of claim 18 further comprising:
hydrogenating said C3-C6 non-aromatic hydrocarbons stream to produce a hydrogenated stream; and
recycling said hydrogenated stream to the reactor.
